(19) United States
(12) Patent Application Publication
(10) Pub. No.: US 2021/0106468 A1
(43) Pub. Date: Apr. 15, 2021

(54) SCHEDULE IDENTIFYING MEDICAL DRESSING

(71) Applicant: RosaMaria Elizabeth Mathews, Rancho Cucamonga, CA (US)

(72) Inventor: RosaMaria Elizabeth Mathews, Rancho Cucamonga, CA (US)

(21) Appl. No.: 17/063,286

(22) Filed: Oct. 5, 2020

Related U.S. Application Data
(60) Provisional application No. 62/913,889, filed on Oct. 11, 2019.

(51) Int. Cl. A61F 13/00; A61M 25/02

(52) U.S. Cl. CPC A61F 13/00059; A61M 25/02; A61M 2205/584; A61F 2013/00412; A61M 2025/0273

(57) ABSTRACT

Methods, systems, and apparatus for a medical dressing. The medical dressing includes a main body that is configured to cover and hold in place a medical device inserted at an insertion site. The medical dressing includes a secure tab that is configured to secure the inserted medical device at the insertion site. The medical dressing includes a label that allows a schedule or a date to be filled-in. The medical dressing includes an indicator that indicates when to remove the medical device.

### CROSS-REFERENCE TO RELATED APPLICATIONS

This application claims priority to and the benefit of U.S. Provisional Patent Application No. 62/913,889 titled “SCHEDULE IDENTIFYING MEDICAL DRESSING,” filed on Oct. 11, 2019, and the entirety of which is hereby incorporated by reference herein.

### BACKGROUND

### 1. Field

This specification relates to a system, apparatus and/or a method for identifying when to remove and/or change a medical dressing.

### 2. Description of the Related Art

Medical dressings have a number of purposes, depending on the type, severity and position of the wound, although all purposes are focused on promoting recovery and protecting the treatment site from further harm. The wound, for example, may have been caused by the insertion of a medical device, such as an intravenous (IV) catheter or needle. The aim of the medical dressing is to promote healing of the wound by providing a sterile environment that facilitates granulation and epithelialization. The medical dressing is often designed to be in direct contact with the wound and may hold or position a catheter, a needle or other medical device within an insertion site. The medical dressing may, for example, help seal the wound to expedite the clotting process, prevent infection to the wound from germs or other foreign objects, and/or soak up blood, plasma and/or other bodily fluids that are exuded from the wound and containing the liquid in one place and preventing maceration.

The catheter, needle or other medical device inserted within the insertion site along with the medical dressing may or may not need to be removed and/or replaced within a timeframe. Generally, however, medical dressings do not readily indicate when the medical dressing and/or the catheter, needle or other medical device may need to be removed and/or replaced. And thus, health professionals, technicians, patients and/or other caretakers fail to identify when to remove and/or replace the catheter, needle or other medical device and/or the medical dressing that may need to be removed and/or replaced. In some instances, a medical dressing that is used to cover and/or protect an insertion site has a label that may be filled with information to indicate when the medical device was inserted into the skin of the patient at the insertion site. This label, however, may be difficult to view and require the health professional, technician or attendant to fill-in information. Moreover, the filled-in information may indicate when the medical device was inserted into the insertion site, which does not indicate when to remove the inserted device and is time-consuming and may result in errors in a hectic environment.

Moreover, different medical procedures require that the inserted device remain in place for different periods of time. And in a hectic environment, such as a hospital, there is not sufficient time or care employed to fill-in the information in the label. Moreover, there is not sufficient time to determine when to remove the inserted device when the filled-in information on the label only indicates the schedule or date of when the medical device was inserted. As a result, an inserted device that is not appropriately removed or replaced on schedule because the nurse, doctor, technician or other had difficulty in easily determining when to make the change may result in additional health consequences.

Accordingly, there is a need for a system, an apparatus and/or a method to more easily identify and/or indicate when the medical dressing and/or the catheter, needle or other medical device inserted should be removed and/or replaced.

### SUMMARY

In general, one aspect of the subject matter described in this specification is embodied in a medical dressing. The medical dressing includes a main body that is configured to cover and hold in place a medical device inserted at an insertion site. The medical dressing includes a secure tab that is configured to secure the inserted medical device at the insertion site. The medical dressing includes a label that allows a schedule or a date to be filled in. The medical dressing includes an indicator that indicates when to remove the inserted medical device.

These and other embodiments may optionally include one or more of the following features. The indicator may include at least one of a schema, pattern, logo or color that indicates the schedule or the date of when to remove the inserted medical device. The at least one of the schema, pattern, or color may include a first schema, pattern or color and a second schema, pattern or color. The first schema, pattern or color may be associated with a first date, day of week or time period to remove the inserted medical device and the second schema, pattern or color may be associated with a second date, day of week or time period to remove the inserted medical device. The first schema, pattern or color may be a first color. The first color may be associated with a first day of week. The second schema, pattern or color may be a second color. The second color may be associated with a second day of week.

The label may occupy, be on or be part of a first area of the main body or the secure tab. The indicator may occupy, be on or be part of a second area of the main body or the secure tab. The second area may be greater than the first area. The indicator may be a colored dye and may be configured to fade over a time period. The indicator may become clear, white, transparent or other default color over the time period to indicate when to remove or replace the inserted medical device. The colored dye may be ingrained within the main body or the secure tab.

The medical dressing may include a transparent window. The transport window may be centered within the main body. The transparent window may be configured to be positioned over the insertion site and allow viewing of a tip of the medical device that is inserted into the insertion site. The medical dressing may include a slot. The slot may be positioned at a distal portion of the main body. The slot may be configured to surround a distal portion of the medical device that is inserted into the insertion site. The slot may center or hold the medical device in place when inserted. The medical dressing may include a layer of adhesive on an edge or perimeter of the slot to form a seal around the insertion site. The layer of adhesive may be transparent and adhere to a skin of the patient. The layer of adhesive may be semi-permeable to allow moisture to escape.

In another aspect, the subject matter is embodied in a medical dressing. The medical dressing includes a main body. The main body is configured to cover and hold in place a device inserted at an insertion site. The medical dressing includes a secure tab that is configured to secure the device at the insertion site. The medical dressing includes a layer of adhesive on the main body and the secure tab. The layer of adhesive is configured to allow the main body and the secure tab to be peeled away from a thin film and to be adhered to a skin of a patient. The medical dressing includes an indicator that indicates when to remove the device.

In another aspect, the subject matter is embodied in a medical dressing. The medical dressing includes a main body. The main body is configured to cover and hold in place an intravenous (IV) catheter inserted at an insertion site. The medical dressing includes a secure tab that is configured to secure the IV catheter at the insertion site. The medical dressing includes a label that allows a schedule or a date to be filled-in. The medical dressing includes a transparent window centered within the main body and configured to be positioned over the insertion site and allow viewing of the device that is inserted into the insertion site. The medical dressing includes a slot positioned at a distal portion of the main body and configured to surround a distal portion of the IV catheter that is inserted into the insertion site. The medical dressing includes an indicator that indicates when to remove the IV catheter.

### BRIEF DESCRIPTION OF THE DRAWINGS

Other systems, methods, features, and advantages of the present invention will be apparent to one skilled in the art upon examination of the following figures and detailed description. Component parts shown in the drawings are not necessarily to scale, and may be exaggerated to better illustrate the important features of the present invention.

FIG. 1A shows an example of a schedule identifying medical dressing using a first indicator to indicate when to replace and/or remove the inserted device according to an aspect of the invention.

FIG. 1B shows an example of a schedule identifying medical dressing using a second indicator to indicate a different timeframe or day of when to replace and/or remove the inserted device according to an aspect of the invention.

FIG. 1C shows an example of a schedule identifying medical dressing using a third indicator to indicate another timeframe or day of when to replace and/or remove the inserted device according to an aspect of the invention.

FIG. 1D shows an example of a schedule identifying medical dressing using multiple different indicators to indicate different information, such as a condition of the patient and/or when to replace and/or remove the inserted device, according to an aspect of the invention.

FIG. 2 shows an example of a schedule identifying medical dressing having a secure attachment device to secure the inserted device in position according to an aspect of the invention.

FIG. 3 shows an example of a schedule identifying medical dressing that is adjustable in size to fit around different size treatment areas according to an aspect of the invention.

FIG. 4 shows an example of a schedule identifying medical dressing that is designed to be easily peelable from the thin film according to an aspect of the invention.

FIG. 5 shows an example of a schedule identifying medical dressing having wings according to an aspect of the invention.

FIG. 6 shows a flow diagram of an example process for attaching the schedule identifying medical dressing according to an aspect of the invention.

### DETAILED DESCRIPTION

Disclosed herein are system, apparatus, devices and/or methods for a schedule identifying medical dressing (or “medical dressing”) that identifies when an intravenous (IV) needle, catheter, syringe, angiocath, angio or other medical device should be removed and/or replaced from an insertion site (hereinafter the IV needle, catheter, syringe, angiocath or other medical device may be referred to as an “inserted device” when inserted into the insertion site). The nurse, doctor, or other health professional, technician or practitioner (or “practitioner”) may identify or determine the insertion site where the practitioner inserts the medical device into the skin of the patient. The insertion site may be a vessel within a body, such as an arm or leg, of a patient. The practitioner may identify the particular medical dressing to apply on top of the inserted device and onto the skin of the patient. The practitioner may peel the medical dressing from its packaging, such as a thin film and place and/or position the medical dressing on top of the skin of the patient and over the inserted device. The inserted device may need to be replaced and/or removed from the insertion site after a period of time, such as a few days or a week after insertion, and/or may need to be replaced and/or removed on a regular or periodic basis.

The medical dressing advantageously indicates in a quicker and more efficient manner that the inserted device may need to be replaced and/or removed by a nurse, doctor, technician or other practitioner than a label with the schedule or the date. The medical dressing may use various colors, labels, logos, patterns and/or other markings or components to more quickly and efficiently identify to a hospital technician, nurse or other healthcare professional that the inserted device should be removed and/or replaced. Since the indicator is very apparent due to its size, schema, pattern, logo, color or code, this reduces the amount of hospital error associated with the changing of inserted devices.

Other benefits and advantages include the capability to be designed to be one-size fits all. For example, the medical dressing may be designed to accommodate inserted devices of various sizes to be positioned on various patients of differing ages and/or sizes. Moreover, in another example, the medical dressing may be designed to adhere to and/or wrap around the treatment site, such as around an arm or other extremity of the patient, to prevent easy removal of the medical dressing, which may expose the treatment location and/or cause the inserted device to dislodge from the insertion site. Additionally, the medical dressing may have one or more markings to facilitate the placement of the medical dressing onto the treatment site and/or location.

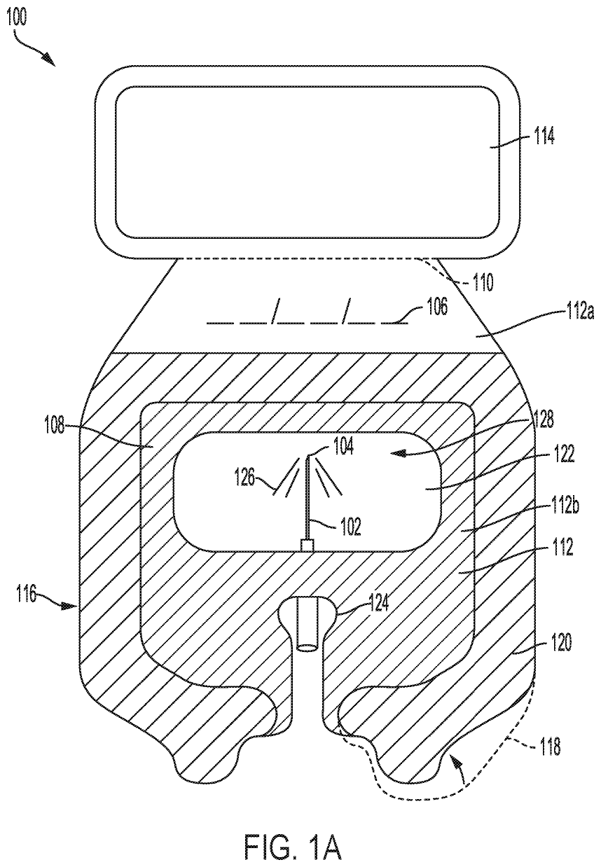

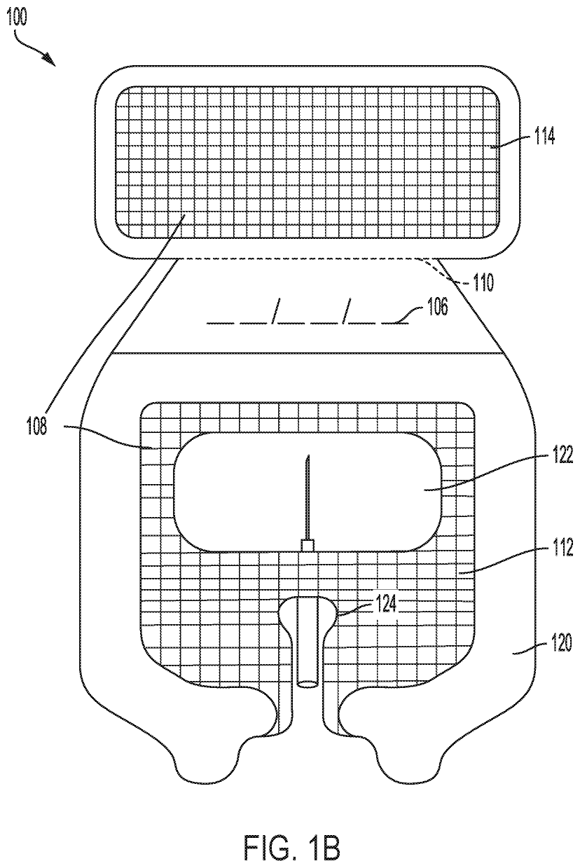

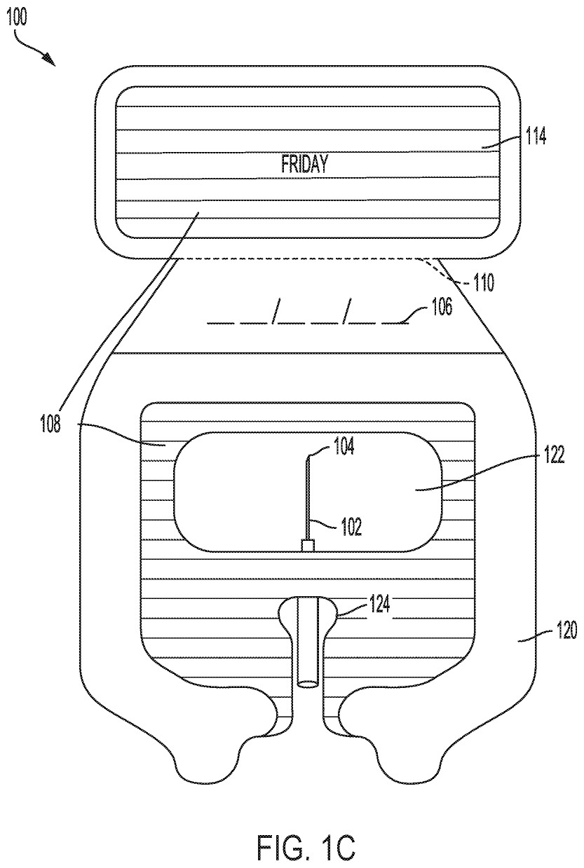

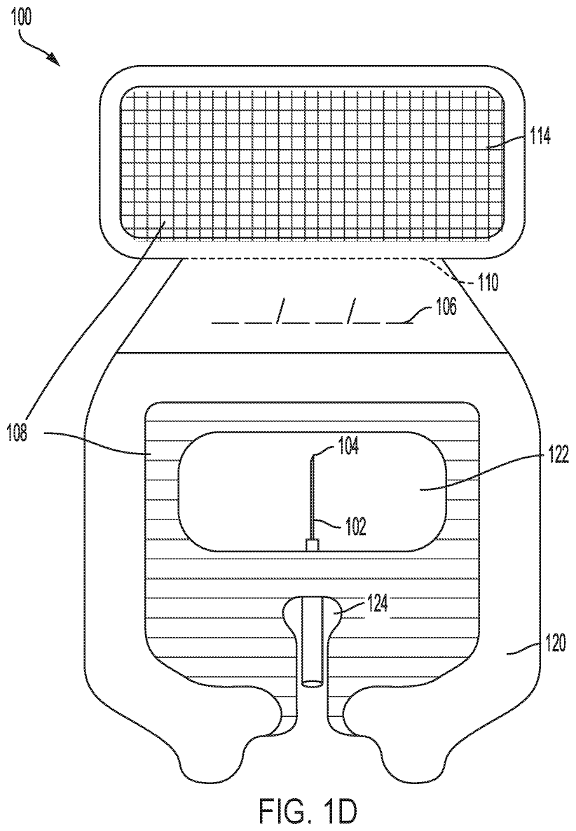

FIGS. 1A-1D show the medical dressing 100 with different types of indicators to indicate when to remove, replace and/or change an inserted device 102 and/or the medical dressing 100. The medical dressing 100 may hold the inserted device 102 in place at the insertion site 104, which prevents movement of the inserted device 102 from the insertion site 104 and/or within the treatment location, e.g., the surrounding area around the insertion site 104. The medical dressing 100 may have a single portion or multiple portions, such as two portions: a main portion or body 112 and a secure tab or portion 114. The different portions may be made from material that is latex-free, water-proof and/or water-resistant to ensure that the treatment site is protected from the external environment. Moreover, the different portions may be semi-permeable and/or semi-porous to be breathable to facilitate healing of the treatment site by allowing some moisture to escape and not be trapped within. The material may also be flexible and/or pliable so that the medical dressing 100 may be shaped around the arm, the extremity or other target area of the patient.

The main portion or body 112 may be formed in a semi-rectangular or square shape with rounded edges or other polygonal shape. The main portion or body 112 may have a cut-out that forms the slot 124 toward a distal portion of the main portion or body 112. The main portion or body 112 may have an upper surface that includes an indicator 108 that indicates when to remove and/or replace the inserted device 102 and/or the medical dressing 100 and a lower surface that has a layer of adhesive 116 to adhere to the skin of the patient and/or the thin film 118. The main portion or body 112 covers the inserted device 102 and surrounds the insertion site to heal the wound caused by the insertion of the medical device and holds the inserted device in a fixed position at the insertion site.

The secure tab or portion 114 may be shaped as a rectangle or other polygon with rounded edges. The secure tab or portion 114 may have a width that is substantially the same as or greater than a width of the main portion or body 112. The secure tab or portion 114 may be initially coupled to the main portion or body 112 but be removable so that the secure tab or portion 114 may be used to secure the main portion or body 112 to the skin of the patient.

The medical dressing 100 may have a perforation 110 that allows separation of the main portion or body 112 from the secure tab or portion 114. In some implementations, there is no perforation 110 but the material of the medical dressing 100 may allow for easy separation. The main portion or body 112 may be affixed or secured to the skin on top of and/or surrounding the insertion site 104 where the inserted device 102 is affixed. The secure tab or portion 114 may be separated from the main portion or body 112 and then may be affixed or secured to the skin or the main portion or body 112 over a portion of the inserted device 102 to secure the inserted device 102 in place against the skin or the main portion or body 112 around the insertion site 104. The main portion or body 112 and the secure tab or portion 114 may be formed from a breathable skin, membrane or dressing that may be porous to allow air to flow through the breathable skin, membrane or dressing between the skin and environment. The breathable skin, membrane or dressing may be made from a cloth or other dressing.

The medical dressing 100 may have a layer of adhesive 116 or other fastener. The layer of adhesive 116 may be on the perimeter and/or on the surface of the main portion or body 112, the window 122, the thin frame 120 and/or the secure tab or portion 114 to form an air-tight or substantially air-tight seal. The layer of adhesive 116 may also be around the slot 124 so that the slot 124 snugly fits around or conforms around the inserted device 102. The layer of adhesive 116 allows the medical dressing 100 to be fastened and secured to the skin surrounding the insertion site 104.

The medical dressing 100 may have a thin film 118 and a thin frame 120, such as a paper liner. The thin frame 120 may surround or border the main portion or body 112 and have a layer of adhesive 116 to stick to the thin film 118. A portion of and/or the entirety of the thin frame 120 may be placed on a surface of the main portion or body 112 that is not in contact with the skin. And as such, the portion of and/or the entirety of the thin frame 120 may use the layer of adhesive 116 to adhere to the surface of the main portion or body 112. Thus, the main portion or body 112 is in between the thin frame 120 and the thin film 118 when the main portion or body 112 has not been peeled away from the thin film 118. The thin frame 120 may be made of a breathable skin, membrane or other permeable or semi-permeable or porous dressing to allow the medical dressing 100 to breathe and allow air, moisture or gas to escape.

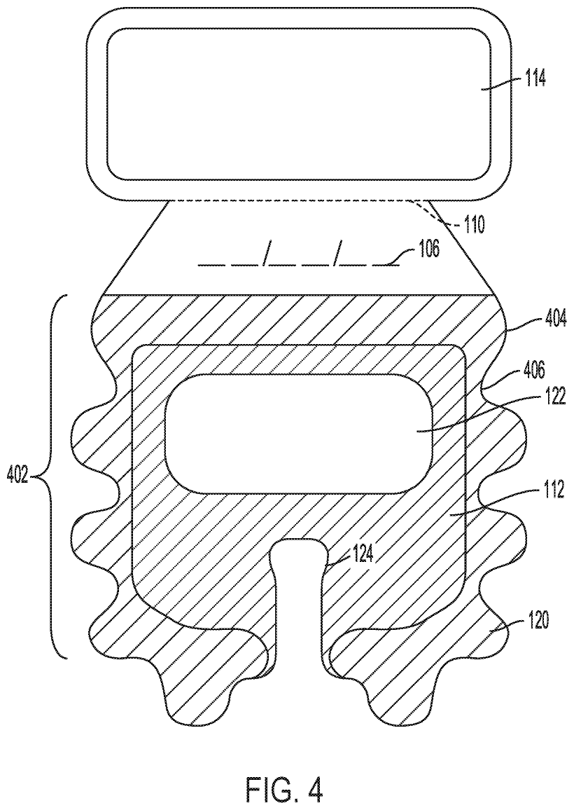

The thin frame 120 may be shaped to surround or border the perimeter of the main portion or body 112. The thin frame 120 may have a portion that extends outward from the main portion or body 112 while keeping the shape of the main portion or body 112 or may be shaped differently, such as in a wavy portion 402, as shown in FIG. 4 for example. The wavy portion 402 may be designed as valleys 406 and peaks 404, which facilitate and allow the thin frame 120 to be easily grasped and removed from the thin film 118 and/or the skin of the patient.

The thin film 118 provides a surface that allows the main portion or body 112, the secure tab or portion 114, the window 122 and/or the thin frame 120 to be peeled away. The main portion or body 112, the window 122, the thin frame 120 and/or the secure tab or portion 114 may initially stick to the thin film 118 using the layer of adhesive 116 during manufacturing, distribution and/or storage before use when the medical dressing 100 is not placed on the skin surrounding the insertion site 104. And subsequently, the main portion or body 112, the window, the thin frame 120 and/or the secure tab or portion 114 may be removed from the thin film 118 to be placed or stuck to the skin surrounding the insertion site 104.

The medical dressing 100 may have a window 122, which may be transparent or semi-transparent window. The window 122 may be positioned at a proximal portion or a centered portion of the main portion or body 112. This window 122 may be placed or positioned over the insertion site 104 and allow viewing of the tip or proximal portion of the inserted device 102 that is inserted into the insertion site 104 and/or viewing of the surrounding area. Hereinafter throughout this specification, distal may refer to away from the insertion site 104 and proximal may refer to situated toward the insertion site 104. This allows a practitioner to monitor the location of the insertion site 104 and/or identify the status of the wound caused by the insertion of the inserted device 102.

The window 122 may have one or more markings 126 on the window 122. The one or more markings 126 may indicate a location to place the ends of the one or more markings 126 over the insertion site and/or the tip of the inserted device 102. When the one or more markings 126 that indicate the location are positioned on top of the tip of the inserted device 102 and/or insertion site 104, this ensures that the medical dressing 100 is uniformly positioned on the inserted device 102 and/or insertion site 104 such that the tip of the inserted device 102 is centered. When the medical dressing 100 is uniformly positioned on top of the inserted device 102, this ensures the medical dressing 100 securely surrounds and encloses the inserted device 102 around the insertion site 104.

In some implementations, the medical dressing 100 may have a layer of an anti-microbial solution or film 128, such as silver. The layer of the anti-microbial solution or film 128 may be on a bottom surface of the window 122. The layer of the anti-microbial solution or film 128 may be used to prevent infection at the insertion site 104. As the anti-microbial solution or film 128 interacts with the germs or other pathogens, the medical dressing 100 may indicate to the nurse, doctor, healthcare professional or practitioner that the inserted device 102 needs to be removed and/or replaced.

The medical dressing 100 may have a slot 124. The slot 124 may be positioned at a distal portion of the main portion or body 112 and surround a distal portion of the inserted device 102 that is inserted into the insertion site 104. The slot 124 may act as a guide for placement of the medical dressing 100 on top of the inserted device 102 and assist in centering or holding in place the inserted device 102. For example, the slot 124 may be shaped with a wider proximal portion than distal portion to facilitate and visually guide placement of the medical dressing 100 on top of the inserted device 102 and to snugly fit and/or conform to the shape of the inserted device 102. The wider proximal portion may fit around and allow the wider end of the inserted device 102 to protrude outward. In some implementations, the slot may be triangularly-shaped, conically-shaped or otherwise shaped, such as in an hourglass shape.

The medical dressing 100 may have a label 106. The label 106 may be pre-filled or filled in at a later time to indicate a schedule or date, such as a month/day/year and/or time. The schedule or the date may refer to when the medical device was inserted into the insertion site 104. In some implementations, the schedule or the date may refer to when to remove and/or replace the medical dressing 100 from the skin surrounding the insertion site 104 and/or remove and/or replace the inserted device 102. In some implementations, the label 106 may have a portion that has one or more spaces or an area that is not pre-filled and necessitates the practitioner to fill-in or otherwise write information into the one or more spaces or the area to indicate when the medical device was inserted and/or when to remove and/or replace the inserted device 102. The label 106 may be positioned within a first area 112a of the main portion or body 112, the secure tab or portion 114 and/or in between the main portion or body 112 and the secure table or portion 114.

The medical dressing 100 may have an indicator 108. The indicator 108 may be different than the label 106. The indicator 108 may indicate a schedule, a date, a day of week or a timeframe (hereinafter referred to as “schedule or date”) of when to remove the inserted device 102 from the skin. The indicator 108 may be pre-filled or printed with information 130, such as a day of the week to remove the inserted device 102, as shown in FIG. 1C for example, or may otherwise use a schema, pattern, logo or color to indicate when the inserted device 102 should be removed without the need for a nurse, hospital technician or professional or other user to fill-in information to indicate when to remove the inserted device 102, which may be required in the label 106. Moreover, the indicator 108 indicates when to remove and/or replace the inserted device 102, whereas, the label 106 may only indicate when the medical device was inserted into the insertion site 104.

The indicator 108 may be positioned on or included within the main portion or body 112 and/or on the secure tab or portion 114. The indicator 108 may include different schemas, patterns, logos, colors or other codes that occupy, are formed within or are otherwise located within a second area 112b of the main portion or body 112 and/or the secure tab or portion 114. The different colors may include red, green, yellow, blue, purple or any other color. The second area 112b may be greater than the first area 112a so that the schemas, patterns, colors or other codes are larger and more visible than the label 106, which makes the schedule or date of when to remove the medical dressing 100 and/or the inserted device 102 more apparent. The schemas, patterns, colors or other codes may occupy, are formed within or are located over the entire main portion or body 112 and/or the entire secure tab or portion 114, which allows the schemas, patterns, colors or other codes to be easily recognizable and visually prominent.

Each medical dressing 100 may have or be associated with a particular or specific schema, pattern, color, logo or other code. Each schema, pattern, color, logo or other code may be different than the other schemas, patterns, colors, logos or other codes, and thus, represent a different schedule, date, day of week or time to remove the inserted device 102. When packaged and sold, the package may contain multiple medical dressings 100 that have different schemas, patterns, colors, logos, or other codes that are associated with the different days of the week or times of when to remove the inserted device 102 and/or associated with the different conditions or diagnosis of the patient. In a set of medical dressings 100, each medical dressing 100 may have a same and/or a different schema, pattern, such as a grid, diagonal, vertical, or horizontal line pattern, color, such as red, blue, green, yellow, purple or other color, or other code than another medical dressing 100 in the set.

For example, a medical dressing 100 may have a blue color or a diagonal line pattern, as shown in FIG. 1A, which indicates that the inserted device 102 must be replaced on a specific day of the week, such as Monday, for example, and another medical dressing 100 may have a red color or a grid, as shown in FIG. 1B, which indicates that the inserted device 102 must be replaced on another day of the week, such as Wednesday, for example. In another example, a horizontal line pattern, as shown in 1C, may indicate that the inserted device 102 must be replaced on another day of the week, such as Friday, for example. The different schemas, patterns, colors or other codes may convey other information. For example, the grid pattern, as shown in FIG. 1B, may indicate that a certain medical procedure was done to this patient, whereas, the diagonal pattern, as shown in FIG. 1A, may indicate that a different medical procedure was done to the patient. Different schemas, patterns, colors or other codes convey different information regarding when to remove and/or replace the inserted device 102 and/or other information regarding the diagnosis or medical procedure related to the patient. The indicator 108 on the main portion or body 112 may be different a schema, pattern, color, logo or other code than another indicator on the secure tab or portion 114.

Each different type of indicator 108 and/or the various locations of the indicator 108 may indicate or represent different information. The location of the indicator 108 may also correspond to different types of information. For example, as shown in FIG. 1D, the indicator 108 on the main portion or body 112 may represent the day of the week to remove and/or replace the inserted device 102 and the indicator on the secure tab or portion 114 may represent the diagnosis, medical procedure or condition related to the patient. In another example, the indicator 108 on the secure tab or portion 114 may represent the day of the week to remove and/or replace the inserted device 102 and the indicator on the main portion or body 112 may represent the diagnosis, medical procedure or condition related to the patient.

In some implementations, the indicator 108 may be a color coded dye. The color coded dye may be formed or ingrained within the breathable skin, membrane or other dressing such that the color coded dye fades over time and is designed to become clear, white, transparent or other default color after a number of day or other period of time. Thus, when the color coded dye fades away from the original color to a default color, a nurse, doctor, technician or other practitioner would understand that the inserted device 102 needs to be replaced and/or removed. The color coded dye could fade away after a number of days, such as 4 days or 7 days.

Within a set of medical dressings 100, the different medical dressings 100 may use different color coded dyes and be associated with the need of the inserted device 102 to be replaced and/or removed after a different number of days. The color coded dyes may begin fading once the main portion or body 112 and/or secure tab or portion 114 is removed from the thin film 118 and adhered to the skin surrounding the insertion site 104. For example, when the main portion or body 112 and/or the secure tab or portion 114 is removed from the thin film 118, a chemical within the color coded dye may react with the air to cause the color to begin to fade or otherwise change. In another example, when the main portion or body 112 and/or the secure tab or portion 114 is adhered to the skin of the patient, a chemical within the color coded dye may react with the moisture on the skin to cause the color to begin to fade or otherwise change.

As a result of the position, the size and/or the different schemas, patterns, logos, colors or other codes of the indicator 108 that is associated with a timeframe to remove and/or replace the inserted device 102, without the need for the practitioner to fill-in information on the medical dressing 100. Additionally, the indicator 108 more clearly indicates and provides immediate recognition to the nurse, doctor, technician or other hospital staff that the inserted device 102 needs to be replaced and/or removed. This reduces human error in determining when to remove and/or replace the inserted device 102.

The nurse, doctor, technician or other hospital staff may have a chart or table that associates and maps the different schemas, patterns, logos, colors or other codes of the indicator 108 to the schedule or the date that the inserted device 102 needs to be replaced and/or removed. Once accustomed to the different schemas, patterns, colors, logos or other codes, the chart or table may no longer be needed. By indicating to the nurse, doctor, technician or other hospital staff that the inserted device 102 needs to be replaced and/or removed, the medical dressing 100 prevents the nurse, doctor, technician or other hospital staff from forgetting that the inserted device 102 needs to be replaced and/or removed. This prevents an infection from occurring.

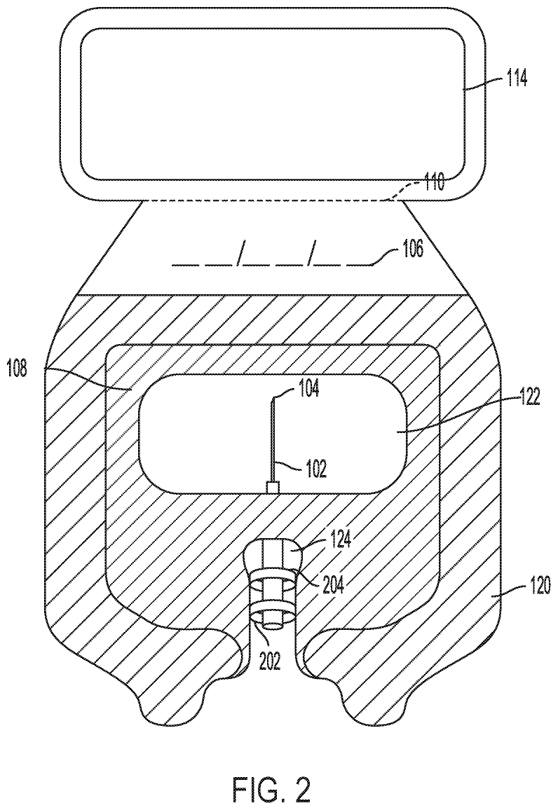

In some implementations, the medical dressing 100 may have one or more clips, fasteners or other attachment devices, such as the clip or fastener 202 as shown in FIG. 2 for example, that are positioned at the distal portion of the main portion or body 112 in proximity to and/or across the slot 124. The attachment device, such as the clip or fastener 202, may be positioned underneath and below the inserted device 102 when the inserted device 102 is snapped-in or otherwise fastened, such that the clip or fastener 202 acts as an under-carriage or supporting frame. The attachment device may be made of a plastic or other polymer and form a casing, housing, enclosure or partial enclosure over, under and/or around a distal portion of the inserted device 102 in a snap-fit arrangement. The attachment device may be molded to conform to the shape of various shapes of inserted devices such that different inserted devices may be snapped into or clipped into the attachment device, such as the clip or fastener 202 in the snap-fit arrangement.

One end of the attachment device, such as the clip or fastener 202, may be affixed to one edge of one side of the slot 124 and the other end may be affixed to another edge of another side of the slot 124. For example, the clip or fastener 202 may be positioned within the slot 124 and may be coupled to edges of the sides of the slot 124 or may have an adhesive that adheres to the skin of the patient. The clip or fastener 202 may receive the distal portion of the inserted device 102 and allow for the inserted device 102 to be snapped into the clip or fastener 202 to hold the inserted device 102 in place after insertion into the insertion site 104. This allows the medical dressing 100 to be placed and arranged over the inserted device 102 that is inserted into the treatment site and may secure the medical dressing 100 over the treatment site and hold the inserted device 102 in place so that the inserted device 102 does not move.

The medical dressing 100 may have a bridge 204 that wraps around or is positioned over the distal portion of the inserted device 102 and the slot 124 to form a bridge across the distal portion of the inserted device 102. One end of the bridge 204 may be positioned along an edge of one side of the slot 124 and wrap around and over the inserted device 102 so that an opposite end of the bridge 204 may couple with another edge of another side of the slot 124. The bridge 204 holds the distal portion of the inserted device 102 against the skin of the patient or the clip or fastener 202 to hold the inserted device 102 in place. The bridge 204 may be a strip of material that works in conjunction with the clip or fastener 202 to secure the inserted device 102.

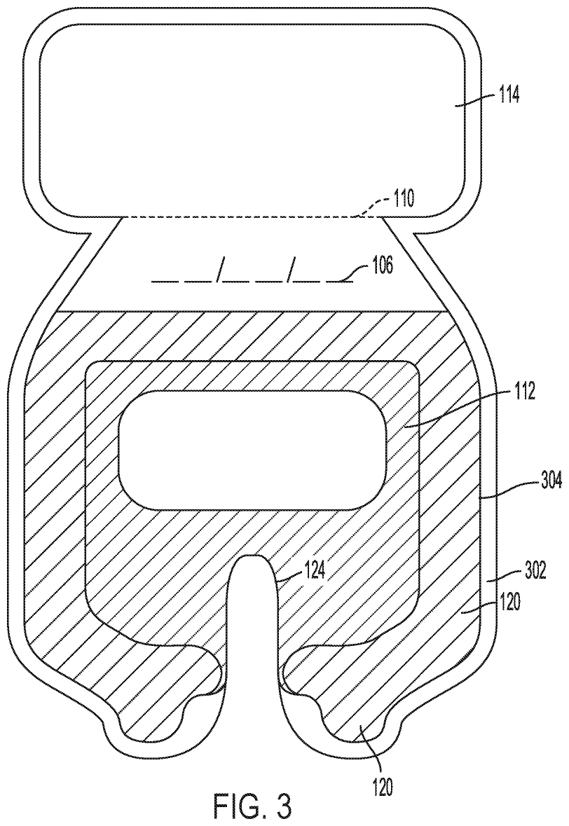

The medical dressing 100 may have an additional outer portion 302 that surrounds the thin frame 120, the main portion or body 112 and the secure tab or portion 114, as shown in FIG. 3 for example. The medical dressing 100 with the additional outer portion 302 may allow the medical dressing 100 to be placed on the skin of patients with different sized treatment locations, such as the arms or legs of the patient. The additional outer portion 302 may provide additional surface area for the medical dressing 100 to adhere to the skin of the patient. The layer of adhesive 116 may extend from the thin frame 120, the main portion or body 112 and/or the secure tab or portion 114 and onto the bottom surface of the additional outer portion 302. Thus, the additional outer portion 302 may provide additional surface area for the medical dressing 100 to adhere to the skin of the patient, such as an adult.

The sticking coefficient of the layer of adhesive 116 may be different for each of the different portions, e.g., the additional outer portion 302, the main portion or body 112, the thin frame 120 and/or the secure tab or portion 114. For example, the sticking coefficient of the layer of adhesive 116 on the additional outer portion 302 may be less than the sticking coefficient of the layer of adhesive 116 on the thin frame 120, which may be less than that of the sticking coefficient of the layer of adhesive 116 on the main portion or body 112 and/or the secure tab or portion 114. Since the outer portions have a lower sticking coefficient than the inner portions, the outer portions, such as the additional outer portion 302 may be more easily peeled or removed to facilitate the removal of the medical dressing 100 because the nurse, healthcare professional or healthcare practitioner removes the outermost edges of the medical dressing to peel the medical dressing 100 inward to remove the medical dressing 100 from either the skin of the patient or the thin film 118.

When the patient is a child or has less surface area on the skin to adhere the medical dressing 100, the medical dressing 100 may have an additional perforation 304 or other edge or area that may be torn or severed and that borders the additional outer portion 302 and interfaces with the thin frame 120, the main portion or body 112 and/or the secure tab or portion 114. The additional perforation 304 allows a user, such as a nurse, doctor, or other health practitioner or professional to remove the additional outer portion 302 that borders the thin frame 120, the main portion or body 112 and/or the secure tab or portion 114. By removing the additional outer portion 302, the medical dressing 100 may be placed on the arm or other treatment location of the patient, which has less surface area to place the medical dressing 100 on. Thus, the medical dressing 100 may be a one-size fits all solution for patients with varying treatment areas, which allows hospitals to purchase a single medical dressing to address patients having different sized treatment areas, which saves costs.

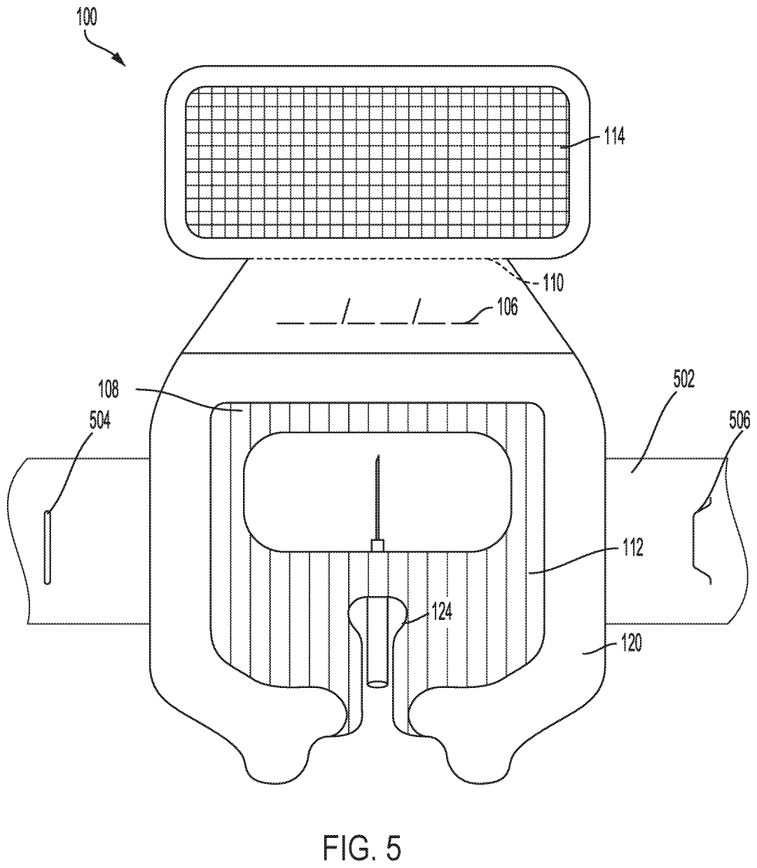

The medical dressing 100 may have one or more wings or phalanges 502, which may extend away from the main portion or body 112 and/or the thin frame 120, as shown in FIG. 5 for example. The one or more wings or phalanges 502 may extend away from the sides of the thin frame 120 such that the one or more wings or phalanges 502 wrap further around the arm or extremity of the patient. This makes the medical dressing 100 more difficult to remove from the skin of the patient. The layer of adhesive 116 may be on a bottom surface of the one or more wings or phalanges to secure and adhere the one or more wings or phalanges 502 to the skin of the patient. In some implementations, the one or more wings or phalanges 502 may have a slit 504 and/or a flap 506. One wing or phalange 502 may have the slit 504 and another wing or phalange 502 on the opposite side of the main portion or body 112 and/or the thin frame 120 may have the flap 506. When the two wings or phalanges 502 are wrapped around the treatment site, the flap 506 may be inserted into the slit 504 to secure the medical dressing 100 around the treatment site to prevent the medical dressing 100 from being easily removed.

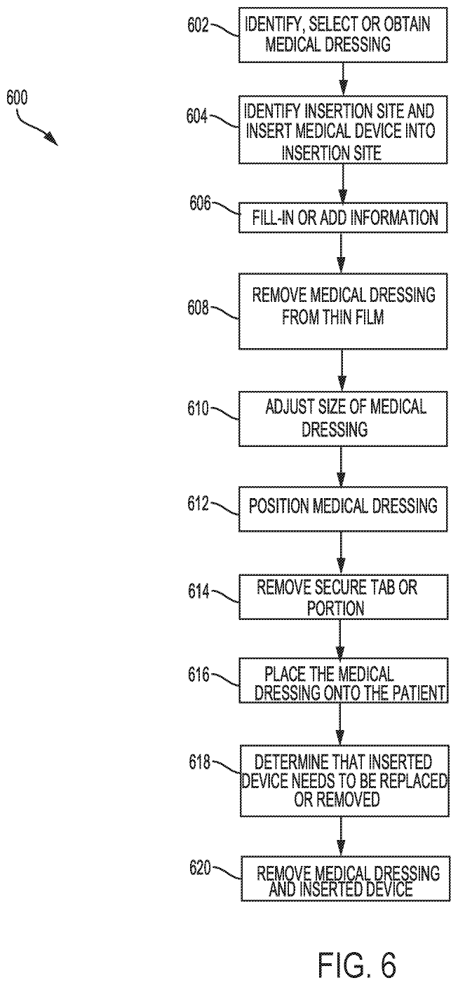

FIG. 6 shows a flow diagram of a process for attaching the medical dressing 100. A practitioner may employ the process to attach the medical dressing 100 and cover the inserted device 102 to protect the insertion site from the environment and hold the inserted device 102 in place until the inserted device 102 needs to be removed and/or replaced.

The practitioner may identify, select and/or obtain the specific medical dressing 100 that needs to be placed on top of the inserted device 102 and to cover the treatment area surrounding the insertion site 104 (602). The practitioner may determine the medical dressing 100 to be used based on the diagnosis and/or condition of the patient and/or when the inserted device 102 needs to be removed and/or replaced Once the practitioner determines when the inserted device 102 needs to be removed and/or replaced and/or the diagnosis and/or condition of the patient, the practitioner may use a chart or other guide to associate the timeframe with when the inserted device 102 needs to be removed and/or replaced along with the diagnosis and/or condition of the patient to a specific medical dressing 100 with the indicator 108 that matches and conveys the timeframe, diagnosis and/or condition of the patient. The specific medical dressing 100 may be selected among various medical dressings in a set of medical dressings that have different indicators to convey different timeframes, diagnosis and/or conditions.

The practitioner identifies an insertion site and inserts the medical device (or inserted device 102) into the insertion site 104 (604). The practitioner may fill-in or add information onto the label 106 to indicate when the medical device was inserted and/or when to remove and/or replace the inserted device 102 (606). The practitioner may add the date, for example, into the one or more spaces or placeholders on the label 106 to indicate when the medical device was inserted and/or when to remove and/or replace the inserted device 102. The practitioner may add other medical information including a diagnosis or condition on the label 106 to inform or remind the practitioner about a condition of the patient.

The practitioner may remove the portions of the medical dressing 100 from the thin film 118 (608). The practitioner may peel-away the main portion or body 112, the secure tab or portion 114, the thin frame 120 and/or other portions of the medical dressing 100 from the thin film 118 so that the medical dressing 100 is at least partially or entirely free from the thin film 118.

The practitioner may adjust a size of the medical dressing 100 (610). The practitioner may adjust the size of the medical dressing 100 based on the size of the treatment area. For example, the practitioner may remove the additional outer portion 302 to reduce the size of the medical dressing on a patient with a smaller treatment area and/or keep the additional outer portion 302 on a patient with a larger treatment area.

The practitioner may position the medical dressing 100 over the insertion site 104 and/or the inserted device 102 (612). The practitioner may use one or markings 126 on the window 122 to guide the positioning and placement of the medical dressing 100 over the inserted device 102. The practitioner may use the one or more markings 126 to align the placement of the window 122 over the treatment area, which aligns the remainder of the medical dressing 100. The window 122 allows the practitioner to view the placement of and adjust the position of the medical dressing 100 on top of the insertion site 104. The practitioner may position the medical dressing 100 over the insertion site 104 while also allowing for the distal portion of the inserted device 102 to be fit on top of the clip or fastener 202 and/or under the bridge 204.

The practitioner, either before or after positioning the medical dressing 100 over the inserted device 102 and/or the insertion site 104, may remove the secure tab or portion 114 from the main portion or body 112 (614). The practitioner by tear or remove the secure tab or portion 114 along the perforation 110 to decouple the secure tab or portion 114 from the main portion or body 112.

When the one or more markings 126 align with the insertion site 104, the practitioner may apply a force on a portion of the medical dressing 100 against the skin of the patient to adhere the portion of the medical dressing 100 onto the skin of the patient (616). The practitioner may adhere the main portion or body 112 onto the skin of the patient and may adhere the secure tab or portion 114 to the main portion or body 112 and/or the skin of the patient. The secure tab or portion 114 may overlap a portion of the main portion or body 112 and/or the skin of the patient to secure the medical dressing to the skin of the patient and/or the inserted device 102 in place. The practitioner may snap or fasten the inserted device 102 into the clip or fastener 202 to hold and secure the inserted device 102 in a fixed position. Then, the practitioner may couple the two or more wings or phalanges 502 to one another, e.g., by wrapping one wing with the flap 506 around the treatment area, such as the arm, and inserting the flap 506 into the slit 504 on another wing, or secure the one or more wings or phalanges 502 around the treatment area using the layer of adhesive 116.

After a period of timeframe indicated by the indicator 108 and/or the label 106 on the medical dressing 100, the practitioner may determine that the inserted device 102 needs to be removed and/or replaced (618). The practitioner may determine that the inserted device 102 needs to be removed and/or replaced based on the one or more indicators 108 and the current date and/or time. The practitioner compares the scheduled date and/or time of removal indicated by the one or more indicators 108 to the current date and/or time, and when the schedule date and/or time of removal matches the current date and/or time, the practitioner may determine that the inserted device 102 needs to be removed and/or replaced.

Once the practitioner determines that the inserted device 102 needs to be removed and/or replaced, the practitioner may remove the medical dressing 100 and/or the inserted device 102 (620). The practitioner may decouple the two or more wings, decouple or unfasten any clips or fasteners, and/or otherwise peel the medical dressing 100 off the skin of the patient to allow for removal of the inserted device 102 from the insertion site 104.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that scope shall not be restricted, except in light of the appended claims and their equivalents.

What is claimed is:

1. A medical dressing, comprising:
a main body that is configured to cover and hold in place a medical device inserted at an insertion site;
a secure tab that is configured to secure the inserted medical device at the insertion site;
a label that allows a schedule or a date to be filled-in; and
an indicator that indicates when to remove the inserted medical device.

2. The medical dressing of claim 1, wherein the indicator includes at least one of a schema, logo, pattern, or color that indicates the schedule or the date of when to remove the inserted medical device.

3. The medical dressing of claim 2, the at least one of the schema, pattern, or color includes a first schema, logo, pattern or color and a second schema, pattern, logo or color, wherein the first schema, pattern, logo or color is associated with a first date, day of week or time period to remove the inserted medical device and the second schema, pattern, logo or color is associated with a second date, day of week or time period to remove the inserted medical device.

4. The medical dressing of claim 3, wherein the first schema, pattern, logo or color is a first color, wherein the first color is associated with a first day of week, wherein the second schema, pattern, logo or color is a second color, wherein the second color is associated with a second day of week.

5. The medical dressing of claim 1, wherein the label occupies, is on or is part of a first area of the main body or the secure tab and the indicator occupies, is on or is part of a second area of the main body or the secure tab, wherein the second area is greater than the first area.

6. The medical dressing of claim 1, wherein the indicator is a colored dye and is configured to fade over a time period and become clear, white, transparent or other default color over the time period to indicate when to remove or replace the inserted medical device.

7. The medical dressing of claim 6, wherein the colored dye is ingrained within the main body or the secure tab.

8. The medical dressing of claim 1, further comprising:
a transparent window centered within the main body and configured to be positioned over the insertion site and allow viewing of a tip of the inserted medical device that is inserted into the insertion site.

9. The medical dressing of claim 1, further comprising:
a slot positioned at a distal portion of the main body and configured to surround a distal portion of the medical device that is inserted into the insertion site, wherein the slot centers or holds the medical device in place when inserted.

10. The medical dressing of claim 9, further comprising:
a layer of adhesive on an edge or perimeter of the slot to form a seal around the insertion site, wherein the layer of adhesive is transparent and adheres to a skin of the patient and is semi-permeable to allow moisture to escape.

11. A medical dressing, comprising:
a main body that is configured to cover and hold in place a device inserted at an insertion site;
a secure tab that is configured to secure the device at the insertion site;
a layer of adhesive on the main body and the secure tab, the layer of adhesive configured to allow the main body and the secure tab to be peeled away from a thin film and to be adhered to a skin of a patient; and
an indicator that indicates when to remove the device.

12. The medical dressing of claim 11, wherein the main body and the secure tab are formed using a breathable skin, membrane, or other dressing that is porous and allows air to flow therethrough.

13. The medical dressing of claim 11, further comprising:
a thin frame that holds the main body against the thin film and assists with peeling the main body from the thin film.

14. The medical dressing of claim 11, wherein the indicator includes at least one of a schema, pattern, or color that indicates the schedule or the date of when to remove the device.

15. The medical dressing of claim 14, the at least one of the schema, pattern, or color includes a first schema, pattern or color and a second schema, pattern or color, wherein the first schema, pattern or color is associated with a first date, day of week or time period to remove the device and the second schema, pattern or color is associated with a second date, day of week or time period to remove the device.

16. The medical dressing of claim 11, wherein the indicator is a colored dye and is configured to fade over a time period and become clear, white, transparent or other default color over the time period to indicate when to remove or replace the device.

17. The medical dressing of claim 16, wherein the colored dye is ingrained within the main body or the secure tab and begins to fade when the main body or the secure tab is in contact with the skin of the patient.

18. The medical dressing of claim 11, further comprising:
a transparent window centered within the main body and configured to be positioned over the insertion site and allow viewing of the device that is inserted into the insertion site.

19. A medical dressing, comprising:
a main body that is configured to cover and hold in place an intravenous (IV) catheter inserted at an insertion site;
a secure tab that is configured to secure the IV catheter at the insertion site;
a label that allows a schedule or a date to be filled-in;
a transparent window centered within the main body and configured to be positioned over the insertion site and allow viewing of the IV catheter that is inserted into the insertion site;
a slot positioned at a distal portion of the main body and configured to surround a distal portion of the IV catheter that is inserted into the insertion site; and
an indicator that indicates when to remove the IV catheter.

20. The medical dressing of claim 19, wherein the indicator includes at least one of a schema, pattern, or color that indicates the schedule or the date of when to remove the IV catheter.
